(19) United States
(12) Patent Application Publication
(10) Pub. No.: US 2014/0364795 A1
(43) Pub. Date: Dec. 11, 2014

(54) SYNTHESIS OF UPCONVERSION NANOCOMPOSITES FOR PHOTODYNAMIC THERAPY

(71) Applicant: Nanyang Technological University, Singapore (SG)

(72) Inventors: Thatt Yang Timothy Tan, Singapore (SG); Qing-Chi Xu, Singapore (SG)

(21) Appl. No.: 14/366,153

(22) PCT Filed: Dec. 17, 2012
(86) PCT No.: PCT/SG2012/000478

§ 371 Date: Jun. 17, 2014

Related U.S. Application Data
(60) Provisional application No. 61/577,477, filed on Dec. 19, 2011.

(51) Int. Cl. A61K 33/24; A61K 49/00; A61N 5/06

(52) U.S. Cl. CPC A61K 33/24; A61N 5/062; A61K 49/005
USPC 604/20; 424/617; 424/9.6

(57) ABSTRACT

The present invention refers to a composite material including at least one upconversion particulate material that under near infrared (NIR) irradiation emits visible light of a wavelength between 380 and 740 nm, and at least one semiconductor particulate material that can absorb the visible light emitted by the at least one upconversion particulate material and upon absorbance generates reactive species, wherein the at least one upconversion particulate material and the at least one semiconductor particulate material are physiologically acceptable. The upconversion particulate material can comprises NaYF4 doped with a one rare earth metal. The bright fluorescence emitted from rare earth-doped NaYF4 upconversion particles is in the visible region of the electromagnetic spectrum and may be absorbed by a biocompatible photocatalysts such as TiO₂ to produce reactive species. This allows the composite to be used for in vivo applications.

### CROSS-REFERENCE TO RELATED APPLICATION

This application claims, the benefit of priority of U.S. application No. 61/577,477 filed Dec. 19, 2011, the contents of it being hereby incorporated by reference in its entirety for all purposes.

### TECHNICAL FIELD

The present invention relates to composites including upconversion particulate materials that convert near infrared (NIR) irradiation into visible light and semiconductor photocatalyst materials that upon absorption of visible light generate reactive species including radicals.

### BACKGROUND

Near infrared-induced drug release and cancer therapy using inorganic nanoparticles have generated much interest because near infrared (NIR) radiation is safe to the body and can penetrate deeper into tissues. Of the various kinds of nanoparticles available, gold (Au) nanoparticles have been utilized in a gold-nanoparticle-mediated hyperthermia system to kill cancer cells and deliver drugs by using NIR laser as an excitation source. Under NIR irradiation, the gold nanoparticles absorb the photon energy and convert it into heat, which raises the temperature of the tissue and eradicate cancer cells by disrupting the cell membrane. Targeted drug delivery based on the NIR-induced photothermal effect of Au nanoparticles has also been reported. Various modifications of Au nanoparticles to enhance the use of Au nanoparticles in these applications have also been undertaken. However, gold nanoparticles are very expensive.

Titanium oxide (TiO₂) nanoparticles are also good candidates for drug delivery and cancer therapy owing to their advantages of high activity, high stability, non-toxicity and low costs. Electron-hole pairs are generated in TiO₂ under ultraviolet (UV) light irradiation and thereby create highly reactive radical oxygen species (ROS). In cancer therapy, ROS can damage the cancer cell membrane and induce programmed cancer cell death, while in drug release, ROS can cleave hydrocarbon chains attached to the surface of TiO₂ and thus lead to the release of drug. However, besides of having the disadvantage of low tissue penetration, the usage of high-energy UV light can cause photo-damage to biological specimens.

It has been reported that YF₃:Yb³⁺,Tm³⁺/TiO₂ core/shell nanoparticles exhibit photocatalytic activity under NIR irradiation due to the photoactivation of TiO₂ by the upconverted UV emission. However, as mentioned above UV light can cause photo-damage to biological specimens and the efficiency of NIR to UV conversion is relatively low. A NaYF₄:Yb,Tm/CdS composite photocatalyst which could degrade Rhodamine B and Methylene blue (MB) under NIR irradiation has also been reported. Unfortunately, cadmium sulfide (CdS) is toxic and unstable, which limits its in vivo applications.

Therefore, there is a need for an improved material for cancer therapy and drug release that is physiologically acceptable, is relatively inexpensive and is more efficient.

### SUMMARY OF THE INVENTION

In a first aspect, the present invention refers to a composite material including at least one upconversion particulate material that under near infrared (NIR) irradiation emits visible light of a wavelength between 380 and 740 nm, and at least one semiconductor particulate material that can absorb the visible light emitted by the at least one upconversion particulate material and upon absorbance generates reactive species, wherein the at least one upconversion particulate material and the at least one semiconductor particulate material are physiologically acceptable.

In a second aspect, the present invention relates to a conjugate including the composite material and at least one compound covalently linked to the composite material.

In a third aspect, the present invention relates to a method for killing cells including contacting said cells with the composite material or the conjugate, and irradiating the cells with the composite material or the conjugate with NIR radiation.

In a fourth aspect, the present invention relates to a method for treating cancer in a subject, including delivering the composite material or conjugate material to said subject and irradiating the subject or part of the subject with NIR radiation.

In a fifth aspect, the present invention relates to a use of the composite or conjugate for the killing of cells.

In a sixth aspect, the present invention relates to a use of the composite or conjugate for the treatment of cancer in a subject.

### BRIEF DESCRIPTION OF THE DRAWINGS

The invention will be better understood with reference to the detailed description when considered in conjunction with the non-limiting examples and the accompanying drawings, in which:

FIG. 1A shows a transmission electron microscopy (TEM) image of NaYF₄:Yb, Tm. The inset of FIG. 1A shows a magnified high resolution transmission electron microscopy (TEM) image NaYF₄:Yb, Tm according to various embodiments of the present invention. FIG. 1B shows a transmission electron microscopy (TEM) image of N—TiO₂/NaYF₄:Yb, Tm. The inset of FIG. 1B shows a magnified high resolution transmission electron microscopy (TEM) image N—TiO₂/NaYF₄:Yb, Tm according to various embodiments of the present invention.

FIG. 2 shows the fourier transform infrared spectra (FTIR) of N—TiO₂, thioglycolic acid modified N—TiO2 (TGA-N—TiO₂), NaYF₄:Yb,Tm and N—TiO₂/NaYF₄:Yb,Tm according to various embodiments of the present invention.

FIG. 3A shows the energy dispersive X-ray spectroscopy (EDX) spectrum of NaYF₄:Yb,Tm according to various embodiments of the present invention arid FIG. 3B shows the energy dispersive X-ray spectroscopy (EDX) spectrum of N—TiO₂/NaYF₄:Yb,Tm according to various embodiments of the present invention.

FIG. 4A shows the X-ray Diffraction Spectra (XRD) of TiO₂, N—TiO₂, NaYF₄:Yb,Tm and N—TiO₂/NaYF₄:Yb,Tm according to various embodiments of the present invention. FIG. 4B shows (i) the NIS X-ray photoelectron spectroscopy (XPS) spectra of N—TiO₂ (ii) a line fitted based on (i) with a peak at 400.0 eV (iii) a line based on (i) with a peak at 396.3 eV.

FIG. 5A is a schematic illustrating visible light emitted by NaYF₄:Yb, Tm being absorbed by doped TiO₂ for a redox reaction to generate reactive oxygen species according to various embodiments of the present invention. FIG. 5B is a schematic illustrating fluorescent dye molecules conjugated with N—TiO₂/NaYF₄:Yb,Tm being released after irradiation with 980 nm laser according to various embodiments of the present invention.

FIG. 6A shows the room temperature emission spectra of (i) nitrogen doped titanium oxide (N—TiO₂) (ii) N—TiO₂/NaYF₄:Yb, Tm according to various embodiments of the present invention (iii) NaYF₄:Yb, Tm. The inset of FIG. 6A shows photographs of light emission of (i), (ii) and (iii). FIG. 6B shows the time-dependent fluorescence spectra of terephthalic acid solution (8×10⁻⁴ M) containing 10 mg of N—TiO₂/NaYF₄:Yb,Tm according to various embodiments of the present invention upon 980 nm NIR irradiation for different periods of time. The inset of FIG. 6B shows a photograph of light emission of the terephthalic acid solution after NIR irradiation. FIG. 6C shows the fluorescence spectra of terephthalic acid solution (8×10⁻⁴ M) containing 10 mg of N—TiO₂/NaYF₄:Yb,Tm according to various embodiments of the present invention as well as control experiments involving NaYF₄:Yb,Tm and N—TiO₂ upon 980 nm NIR irradiation for 120 minutes.

FIG. 7 shows the UV-Visible Light (UV-Vis) diffuse reflectance spectra of the N—TiO₂, NaYF₄:Yb,Tm and N—TiO₂/NaYF₄:Yb,Tm according to various embodiments of the present invention. The inset of FIG. 7 shows the NIR diffuse reflectance spectra of the N—TiO₂, NaYF₄:Yb,Tm and N—TiO₂/NaYF₄:Yb,Tm.

FIG. 8A shows the variation in absorbance spectra of Methylene Blue (MB) catalyzed by N—TiO₂/NaYF₄:Yb,Tm according to various embodiments of the present invention under different periods of NIR irradiation time. FIG. 8B shows the comparison of the normalized concentration of MB decomposed by the N—TiO₂, NaYF₄:Yb,Tm, and N—TiO₂/NaYF₄:Yb,Tm according to various embodiments of the present invention under NIR laser irradiation.

FIG. 9 shows the fluorescence spectra of solution with N—TiO₂/NaYE₄:Yb,Tm according to various embodiments of the present invention removed (i) before NIR irradiation (ii) after NIR irradiation. The inset of FIG. 9 shows a photograph of the solution exhibiting a strong blue fluorescence after NIR irradiation.

FIG. 10 shows formation of a nanoconjugate according to various embodiments of the present invention.

FIG. 11 shows the fourier transform infrared (FTIR) spectra of (a) Dopamine-immobilized nanocomposites (b) 1,1′-carbonyldiimidazole (CDI)-immobilized nanocomposites and (c) antibody-conjugated nanocomposites (nanoconjugates) according to various embodiments of the present invention.

FIG. 12 shows the fourier transform infrared (FTIR) spectra of antibody-conjugated nanocomposites (nanoconjugates) according to various embodiments of the present invention.

FIG. 13 show florescence activated cell sorting (FACS) analysis when cells are treated with anti-cAngpt14 conjugated with nanocomposites to form nanoconjugates according to various embodiments of the present invention.

### DETAILED DESCRIPTION OF THE INVENTION

In the present context, “physiologically acceptable” as used herein refers to having substantially no negative impact on an organism, such as a eukaryotic organism, for example a human or animal, upon administration in a pharmaceutically effective amount. Accordingly, a “physiologically acceptable material” as used herein refers to a material substantially having no negative impact on an organism, in particular a human or animal, upon administration in a pharmaceutically effective amount. In other words, a “physiologically acceptable material” can be introduced into the body of a host without having a toxic effect and without significantly decreasing viability compared to an untreated organism. The physiologically acceptable material may be a non-toxic material and/or a biocompatible material. It is particularly preferred that also potential metabolites of the material exhibit the same non-toxic properties. The afore-mentioned properties of being physiologically acceptable, non-toxic and/or biocompatible may be limited to states where the material is not exposed to NIR radiation, i.e. states where no radical or reactive species formation due to photocatalytic activity occurs. It is of course understood that once the material is irradiated with NIR radiation, the thus generated radicals or reactive species may have toxic effects on nearby cells.

In the present context, “particulate” as used herein refers to having a separate and granular form and “particulate material” refers to a material having a separate and granular form. In the present context, “reactive species” as used herein refers to chemically reactive atoms or molecules capable of causing damage to cell structures and may include radicals, ions, and electronically excited molecules.

In a first aspect, the present invention refers to a composite material including at least one upconversion particulate material that under near infrared (NIR) irradiation emits visible light of a wavelength between 380 and 740 nm, and at least one semiconductor particulate material that can absorb the visible light emitted by the at least one upconversion particulate material and upon absorbance generates reactive species, wherein the at least one upconversion particulate material and the at least one semiconductor particulate material are physiologically acceptable.

In various embodiments, the at least one semiconductor particulate material absorbs the visible light and catalyzes the formation of the reactive species. In various embodiments, the reactive species are formed from water or other suitable substances. The energy of the visible light emitted from the upconversion material absorbed by the at least one semiconductor particulate material is used to generate electrons in the conduction band and holes in the valence band of the semiconductor particulate material. In various embodiments, reactive species may be generated from water or other suitable substances surrounding the at least one semiconductor particulate material by the movement of the generated electrons or/and holes to the water or other suitable molecules. In this manner, the at least one semiconductor particulate material acts as a catalyst in the formation of reactive species. In other words, the at least one semiconductor particulate material upon absorbance of visible light generates reactive species from water or other suitable substances in presence of visible light.

In various embodiments, the upconversion and semiconductor particulate materials may be nanoparticulate materials. Nanoparticulate materials refer to particulate materials that have in their greatest dimension a mean diameter of 100 nm or smaller, preferably in the range of about 1 to about 50 nm. In various embodiments, the composite material may be a nanocomposite material. A nanocomposite material is a composite material formed from nanoparticulate materials.

In various embodiments, the at least one upconversion particulate material and the at least one semiconductor particulate material may be bonded to each other. The at least one upconversion particulate material and the at least one semiconductor particulate material may be bonded to each other via at least one linker molecule. In various embodiments, the at least one upconversion particulate material and the at least one semiconductor particulate material may be bonded to each other directly or via linker molecules. In various embodiments, the bonding includes covalent bonding or interaction. In various embodiments, the bonding includes non-covalent bonding or interaction such as ionic bonding. In various embodiments, the at least one upconversion particulate material and the at least one semiconductor particulate material may be associated with each other. In various other embodiments, the at least one upconversion particulate material and the at least one semiconductor particulate material may not be bonded to each other or associated with each other but are placed in close proximity to each other such that the at least one semiconductor particulate material is able to absorb the visible light emitted from the at least one upconversion particulate material. In various embodiments, the at least one upconversion particulate material and the at least one semiconductor particulate material may be brought in close proximity to each other by deposition, mixing or any other suitable means. In various embodiments, the at least one upconversion particulate material and the at least one semiconductor material may be separated by a distance of less than 200 nm or less than 150 nm or less than 100 nm or less than 50 nm or less than 10 nm or less than 7 nm or less than 5 nm. The linker molecule may be an alkyl group with at least two functional groups, with one example being thioglycolic acid (SHCH₂COOH), thiopropanoic acid, thiobutyric acid, mercaptoethanol, polyethylenimine without being limited thereto. The at least two functional groups may be of different types or of the same type. In various embodiments, the linker molecule may be linear or branched. In various embodiments, the linker molecules may have two or more carbon atoms. A first linker molecule such as thioglycolic acid may be first bonded with the at least one semiconductor particulate material, for example via its thiol group which readily binds to many metals and metal oxides. A second linker molecule such as polyethylenimine or mercaptoethanol may be bonded to the at least one upconversion particulate material. Thioglycolic acid then reacts with plyethylenimine or mercaptoethanol to form a bond. In this manner, the at least one upconversion particulate material is bonded to the at least one semiconductor particulate material. In this manner, linker molecules facilitate bonding between the at least one upconversion particulate material and the at least one semiconductor particulate material.

In various embodiments, the upconversion particulate material comprises or consists of NaYF₄ doped with at least one rare earth metal. Rare earth-doped NaYF₄ upconversion particles emit bright fluorescence (green, blue, etc.) under NIR light excitation. The bright fluorescence emitted from rare earth-doped NaYF₄ upconversion particles is in the visible region of the electromagnetic spectrum and may be absorbed by photocatalysts to produce reactive species. Advantageously, rare earth-doped NaYF₄ upconversion particulate materials are stable and have low cytotoxicity. This allows the composite to be used for in vivo applications.

In various embodiments, the at least one rare earth metal is selected from the group consisting of Sc, Y, La, Ce, Pr, Nd, Pm, Sm, Eu, Gd, Tb, Dy, Ho, Er, Lu, Yb, Tm and combinations thereof.

The upconversion particulate material may be NaYF₄:Yb; NaYF₄:Tm or NaYF₄:Yb, Tm.

In various embodiments, the at least one semiconductor particulate material comprises or consists of TiO₂ doped with another element. TiO₂ is a biocompatible material. Advantageously, this allows the composite to be used for in vivo applications.

The dopant of the semiconductor particulate material may be selected from the group consisting of N, P, C, B, S, Fe, Au, Ce, Er, Eu or any other suitable elements and combinations thereof.

The dopants may be selected such that the energy bandgap of the semiconductor particulate material is able to absorb visible light emitted by the upconversion particulate material in an efficient manner. The energy band gap of undoped TiO₂ is about 3.2 eV and UV light with a wavelength of less than 380 nm is required to activate undoped TiO₂. Doping with a suitable dopant may lower the energy band gap such that visible light of a wavelength between 380 and 740 nm may be absorbed by the TiO₂ for activation. This allows the use of a suitable upconversion particulate material that can convert NIR to visible light. Advantageously, this is more efficient and less damaging to biological tissues compared to NIR to UV conversion.

Alternatively, other physiologically acceptable semiconductor particulate materials that are able to absorb visible light of a wavelength between 380 and 740 nm may be used. Other non-limiting examples include Bi₂WO₆, N—ZnO, SrTiO₃, N—Bi₂O₃, WO₃, CaBi₆O₁₀, Bi₂TiO₄F₂.

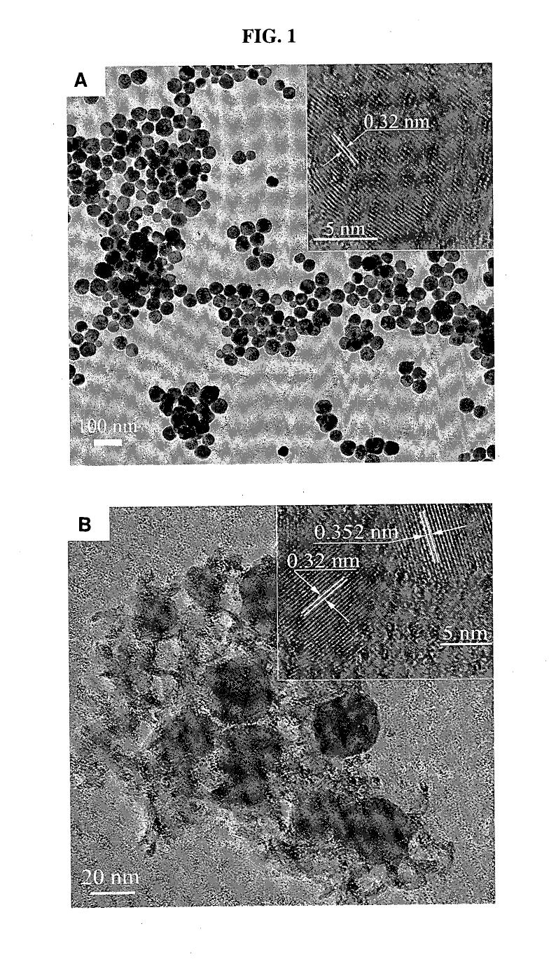

In various embodiments, the upconversion particulate material is NaYF₄:Yb, Tm and the semiconductor particulate material is nitrogen doped TiO₂ (N—TiO₂). FIG. 1A shows a transmission electron microscopy (TEM) image of NaYF₄:Yb, Tm. The inset of FIG. 1A shows a magnified high resolution transmission electron microscopy (TEM) image NaYF₄:Yb, Tm. FIG. 1B shows a transmission electron microscopy (TEM) image of N—TiO₂/NaYF₄:Yb, Tm according to various embodiments of the present invention. The inset of FIG. 1B shows a magnified high resolution transmission electron microscopy (TEM) image N—TiO₂/NaYF₄:Yb, Tm according to various embodiments of the present invention.

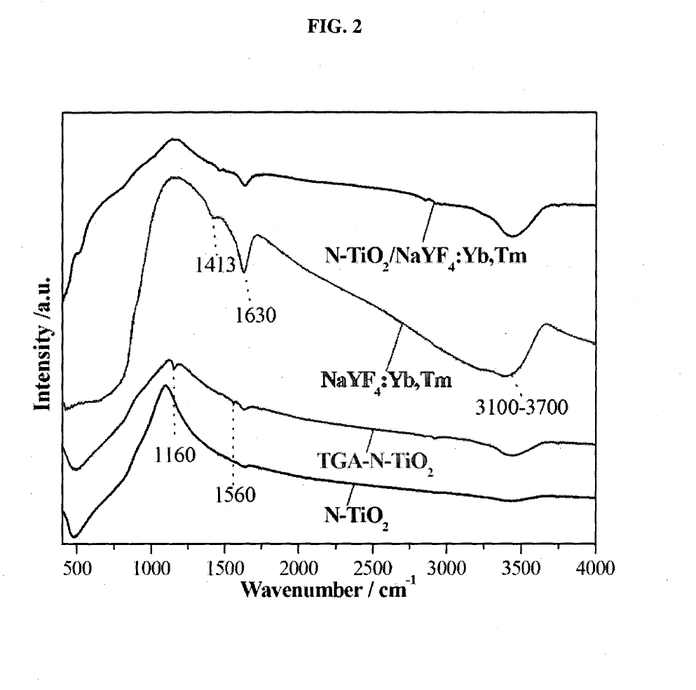

FIG. 2 shows the fourier transform infrared spectra (FTIR) of N—TiO₂, thioglycolic acid modified N—TiO₂ (TGA-N—TiO₂), NaYF₄:Yb,Tm and N—TiO₂/NaYF₄:Yb,Tm according to various embodiments of the present invention. The peaks at 1630 and 3100-3700 cm⁻¹ are attributed to the stretching vibrations of the O—H bending of adsorbed water molecules and O—H absorbed on NaYF₄. The peaks at 1160 and 1560 cm⁻¹ are assigned to the C—O stretching vibration and carboxyl stretching vibration, respectively. FIG. 2 shows the presence of carboxyl and hydroxyl in TGA-N—TiO₂ and NaYF₄:Yb,Tm respectively, which facilitates covalent bonding between N—TiO₂ and NaYF₄:Yb,Tm.

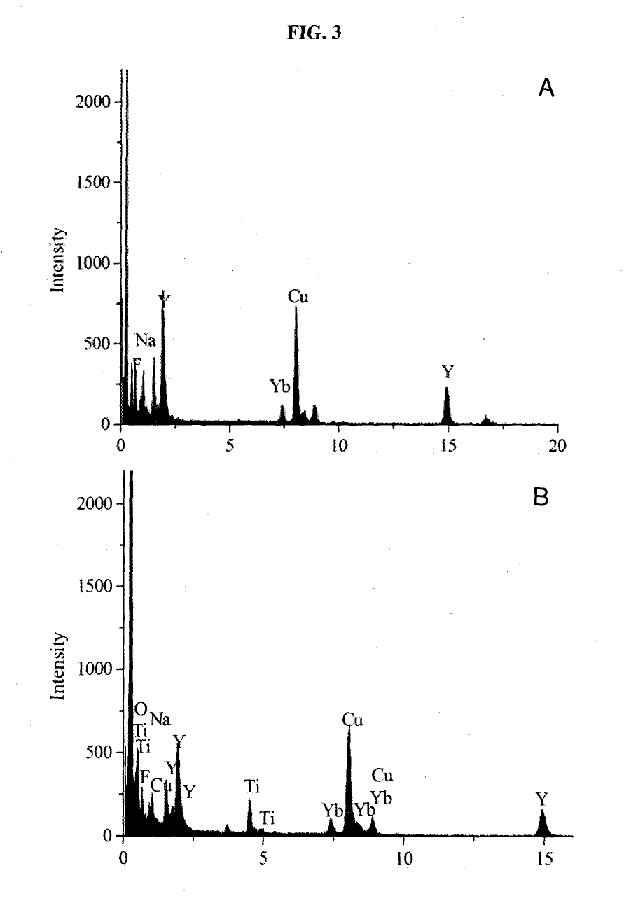

FIG. 3A shows the energy dispersive X-ray spectroscopy (EDX) spectrum of NaYF₄:Yb,Tm and FIG. 3B shows the energy dispersive X-ray spectroscopy (EDX) spectrum of N—TiO₂/NaYF₄:Yb,Tm according to various embodiments of the present invention. FIG. 3B has additional peaks relating to Ti, proving the formation of N—TiO₂/NaYF₄:Yb,Tm.

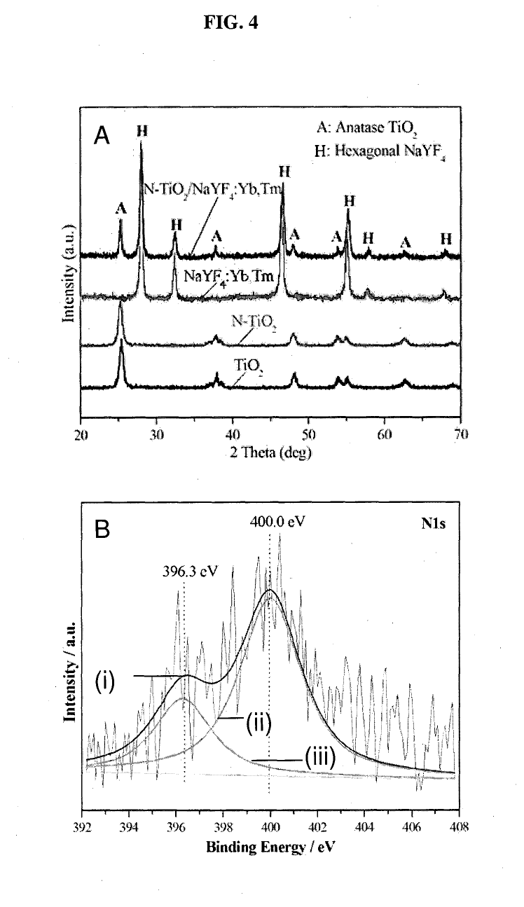

FIG. 4A shows the X-ray Diffraction Spectra (XRD) of TiO₂, N—TiO₂, NaYF₄:Yb,Tm and N—TiO₂/NaYF₄:Yb,Tm according to various embodiments of the present invention. FIG. 4A shows the presence of NaYF₄ crystalline phase and several weak peaks of anatase TiO₂ in N—TiO₂/NaYF₄:Yb,Tm according to various embodiments of the present invention. FIG. 4B shows (i) the N1S X-ray photoelectron spectroscopy (XPS) spectra of N—TiO₂ (ii) a line fitted based on (i) with a peak at 400.0 eV (iii) a line based on (i) with a peak at 396.3 eV. The smaller peak at 396.3 eV suggests the doping of N into the lattice of N—TiO₂.

The reactive species generated by the semiconductor particulate material may be a reactive oxygen species (ROS). Reactive oxygen species (ROS) are chemically reactive atoms or molecules containing oxygen. Reactive oxygen species (ROS) may include radicals such as O. and OH. as well as superoxide anions (O₂.), hydrogen peroxide and singlets oxygen (¹O₂).

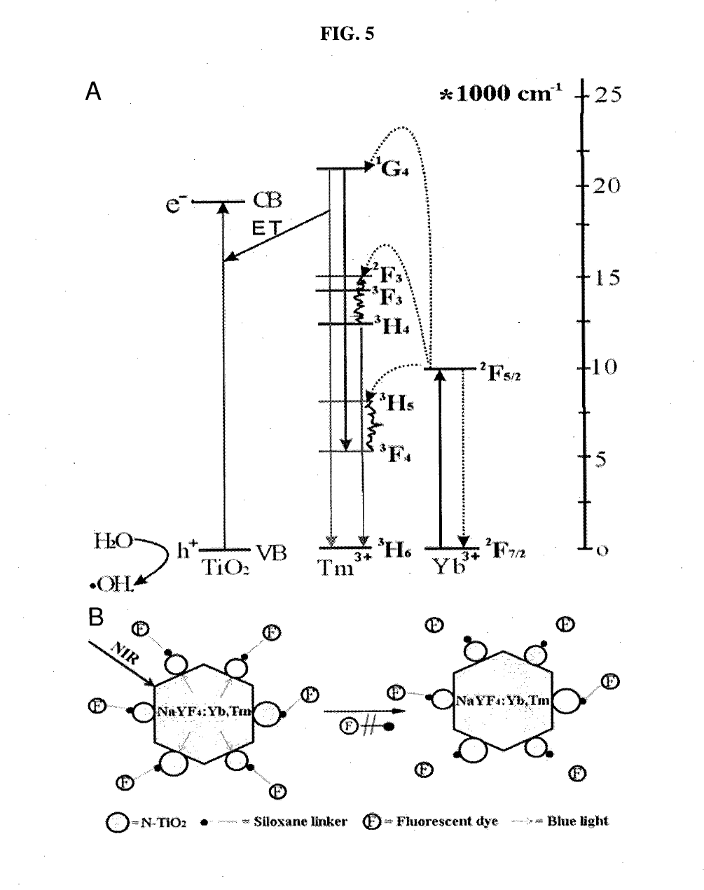

FIG. 5A is a schematic illustrating visible light emitted by NaYF₄:Yb, Tm being absorbed by doped TiO₂ for a redox reaction to generate reactive oxygen species according to various embodiments of the present invention. FIG. 5B illustrates fluorescent dye molecules conjugated on N—TiO₂/NaYF₄:Yb,Tm being released after irradiation with 980 nm laser according to various embodiments of the present invention. Under NIR laser irradiation, the upconversion nanoparticulate material (UCNP), i.e. NaYF₄:Yb,Tm, emit visible light (wavelength=470 nm), which can excite N-doped TiO₂ to generate electron-hole pairs for redox reaction. This occurs when the sensitizers (Yb³⁺) in the NaYF₄:Yb,Tm absorb 980 nm photons due to the NIR laser irradiation and successively transfer their energies to neighboring Tm³⁺ ions. Electrons in the Tm³⁺ ions are excited to the ¹G₄ and ³H₄ states. Then the excited Tm³⁺ ions relax radiatively to ³H₆ or ³H₄ and gives arise to three characteristics emissions at 470, 650 and 800 nm, corresponding to ¹G₄→³H₆, ¹G₄→³H₄ and ³H₄→³H₆ transitions, respectively.

In a second aspect, the present invention relates to a conjugate including the composite material and at least one compound covalently linked to the composite material.

In various embodiments, the conjugate is a conjugate material including a composite material and at least one compound covalently linked to the composite material.

In various embodiments, the conjugate may be a nanoconjugate material. Nanoconjugates refer to conjugates formed by covalently linking a nanocomposite material with at least one compound.

In various embodiments, the at least one compound may be a therapeutic substance. In various embodiments, the at least one compound may be a drug or targeting moiety. In various embodiments, the at least one compound may be selected from the group comprising dyes, proteins, peptides and drugs.

In various embodiments, the drug may be a small molecule drug or an antibody. In various embodiments, the drug may be an anti-cancer drug. The antibody may be a cancer-targeting antibody such as a monoclonal antibody to fibrinogen-like angiopoietin-like 4 (anti-cAngpt114; clone mAb 11F6C4). Other non-limiting examples may include gemtuzumab, alemtuzumab and rituximab.

In various embodiments, the at least one compound is covalently linked to the composite materials through one or more coupling agents. The coupling agents may include but are not limited to dihydroxy-phenylalanine (DOPA), 1,1′-carbonyldiimidazole (CDI) and 3-aminopropytriethoxysilane.

FIG. 5B illustrates a dye, 7-methoxycoumarin-3-carboxylic acid on the surface of N—TiO₂/NaYF₄: Yb,Tm according to various embodiments of the present invention to investigate NIR-induced drug release. 3-aminopropytriethoxysilane is used as a cross-linker. FIG. 5B shows that the 470 emission emitted by the Tm³⁺ ions during relaxation is absorbed by the surrounding N—TiO₂, which leads to the generation of holes in the valence band and electrons in the conduction band of the N—TiO₂, and thereby generating highly reactive radical species (OH.). The highly reactive hydroxyl radicals (OH.) cleave the hydrocarbon chains attached to the surface of the N—TiO₂/NaYF₄:Yb,Tm leading to the release of the 7-methoxycoumarin-3-carboxylic acid. Similarly, other compounds can be attached to the surface of the N—TiO₂/NaYF₄:Yb,Tm and be released in the same manner. In various embodiments, other reactive species may be used to cleave the hydrocarbon bonds.

In this manner, the compound bound to the composite material can be easily released in an efficient manner when the composite material absorbs NIR and generates free radicals or reactive species.

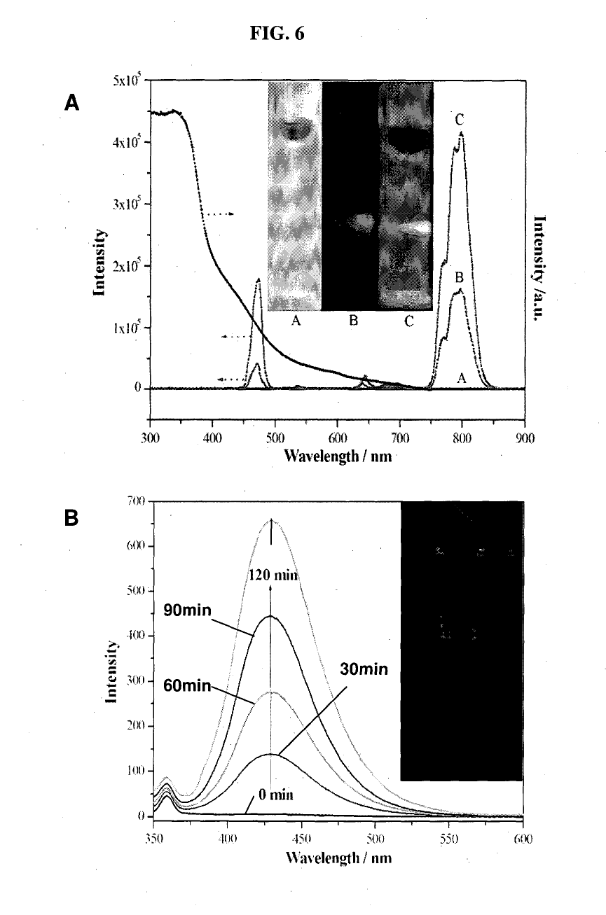

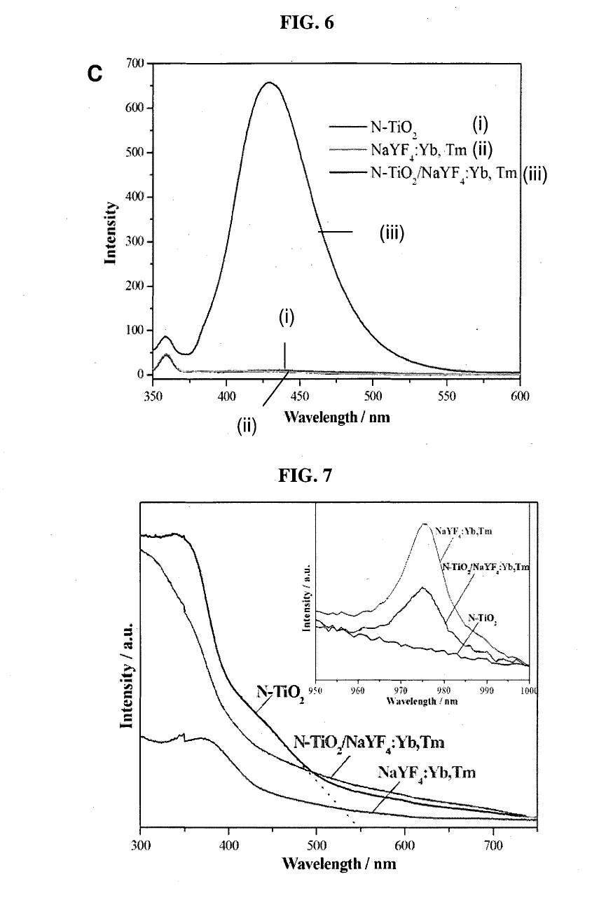

FIG. 6A shows the room temperature emission spectra of (i) nitrogen doped titanium oxide (N—TiO₂) (ii) N—TiO₂/NaYF₄:Yb, Tm according to various embodiments of the present invention (iii) NaYF₄:Yb, Tm. The inset of FIG. 6A shows photographs of light emission of (i), (ii) and (iii). Under NIR irradiation, NaYF₄:Yb,Tm emits blue light with emission peak at 470 nm. As shown in FIG. 6A, the N—TiO₂ exhibits light absorption at 470 nm, indicating its potential to absorb the blue light emission from NaYF₄:Yb,Tm. The photoluminescence (PL) intensity of the N—TiO₂/NaYF₄:Yb,Tm at blue emission peak of 470 nm (I₄₇₀) is much lower than that of NaYF₄:Yb,Tm. The PL intensities of the N—TiO₂/NaYE₄:Yb,Tm at emission peaks of 640 nm and 795 nm (I₇₉₅) are also lower than that of NaYF₄:Yb,Tm, which is probably because the surrounding N—TiO₂ blocks these emissions to some extent. However, the reductions of the emission peaks at 640 nm and 795 nm are much lower than that of the emission peak at 470 nm. The PL intensity ratio of I₄₇₀/I₇₉₅ for the N—TiO₂/NaYF₄:Yb,Tm composite is 0.23, which is much smaller than the value of 0.43 for the pure NaYF₄:Yb,Tm, suggesting that some of the blue emission at 470nm is absorbed by N—TiO₂.

To further prove the energy transfer from NaYF₄:Yb,Tm to N—TiO₂ and the generation of hydroxyl radicals (OH.), a fluorescence analytical technique based on a terephthalic acid (TA) reaction is employed. FIG. 6B shows the time-dependent fluorescence spectra of terephthalic acid solution (8×10⁻⁴ M) containing 10 mg of N—TiO₂/NaYF₄:Yb,Tm according to various embodiments of the present invention upon 980 nm NIR irradiation for different periods of time. The inset of FIG. 6B shows a photograph of light emission of the terephthalic acid solution after NIR irradiation. The fluorescence intensity gradually increases with the increase of irradiation time. The generation of strong blue fluorescence (FIG. 6B inset) upon excitation proves the formation of OH. radicals. FIG. 6C shows the fluorescence spectra of terephthalic acid solution (8×10⁻⁴ M) containing 10 mg of N—TiO₂/NaYF₄:Yb,Tm according to various embodiments of the present invention as well as control experiments involving NaYF₄:Yb,Tm and N—TiO₂ upon 980 nm NIR irradiation for 120 minutes. No fluorescence can be seen for the control experiments involving NaYF₄:Yb,Tm and N—TiO₂ under NIR irradiation.

FIG. 7 shows the UV-Visible Light (UV-Vis) diffuse reflectance spectra of the N—TiO₂, NaYF₄:Yb,Tm and N—TiO₂/NaYF₄:Yb,Tm according to various embodiments of the present invention. The inset of FIG. 7 shows the NIR diffuse reflectance spectra of the N—TiO₂, NaYF₄:Yb,Tm and N—TiO₂/NaYF₄:Yb,Tm. It is observed that NaYF₄:Yb,Tm in NaYF₄:Yb,Tm and N—TiO₂/NaYF₄:Yb,Tm absorbs NIR photons at wavelengths 980 nm. This is because Yb³⁺ has a large absorption cross section of 970 to 1000 nm.

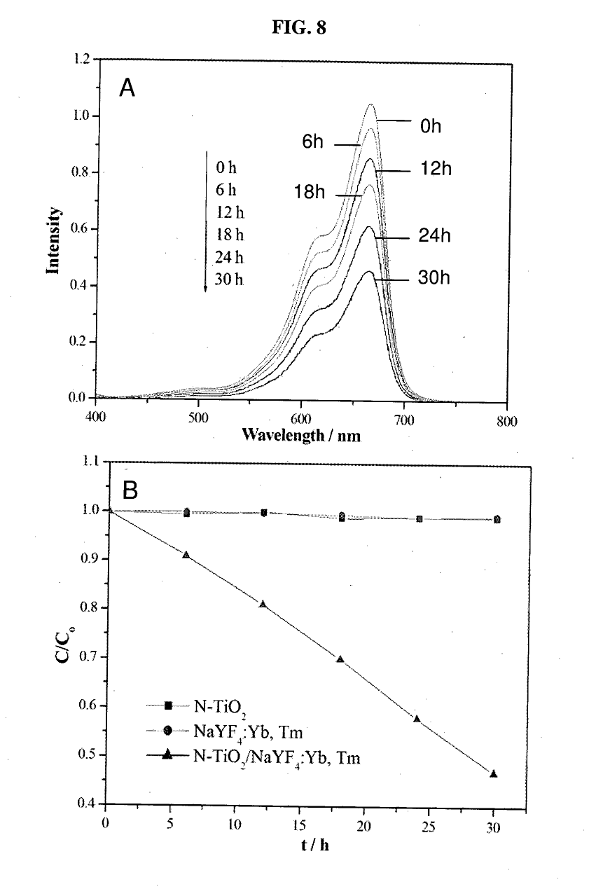

The photocatalytic activity of the N—TiO₂/NaYF₄:Yb,Tm under NIR irradiation was measured using Methylene Blue (MB) as a model organic compound. FIG. 8A shows the variation in absorbance spectra of Methylene Blue (MB) catalyzed by N—TiO₂/NaYF₄:Yb,Tm according to various embodiments of the present invention under different periods of NIR irradiation time. The absorption band at 664 nm decreases with the increase of NIR irradiation time, showing that the N—TiO₂/NaYF₄:Yb,Tm according to various embodiments of the present invention is capable of degrading MB. FIG. 8B shows the comparison of the normalized concentration of MB decomposed by the N—TiO₂, NaYF₄:Yb,Tm, and N—TiO₂/NaYF₄:Yb,Tm according to various embodiments of the present invention under NIR laser irradiation. It was observed that 56% of MB was degraded in the presence of N—TiO₂/NaYF₄:Yb,Tm after 30 h irradiation. However, no obvious degradation of MB was observed in the presence of pure N—TiO₂ or pure NaYF₄:Yb,Tm.

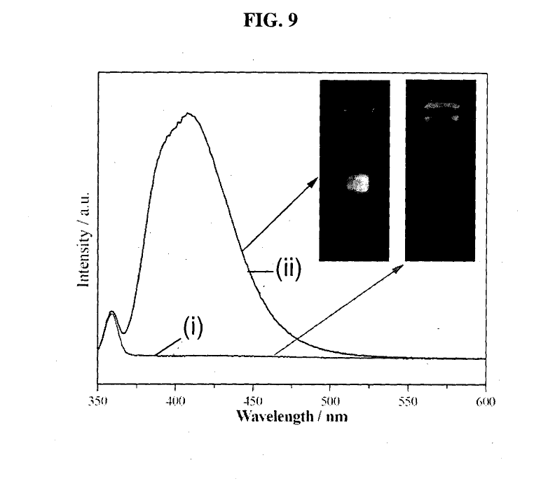

To demonstrate NIR-induced drug release, a model drug (7-methoxycoumarin-3-carboxylic acid) was attached to the N—TiO₂/NaYF₄:Yb,Tm according to various embodiments of the present invention by using 3-aminopropyltriethoxysilane as an intermediate molecule. The N—TiO₂/NaYF₄:Yb,Tm was dispersed into a quartz cuvette containing deionized (DI) water. Before and after NIR irradiation, N—TiO₂/NaYF₄:Yb,Tm was removed and the remaining solution was measured by using PL technique. Under NIR irradiation, cleaving takes place at the anchoring siloxane groups, which causes the release of the model drug into DI water. The results of NIR-induced drug release are shown in FIG. 9. FIG. 9 shows the fluorescence spectra of solution with N—TiO₂/NaYF₄:Yb,Tm removed (i) before NIR irradiation (ii) after NIR irradiation. The inset of FIG. 9 shows a photograph of the solution exhibiting a strong blue fluorescence after NIR irradiation. Before NIR irradiation, there is no fluorescence observed, which indicates that no dye is released. After NIR irradiation, a strong blue fluorescence (FIG. 9 inset) of high intensity peak at 405 nm (excitation wavelength 320 nm) was observed, which proves the release of the model drug into DI water under NIR irradiation.

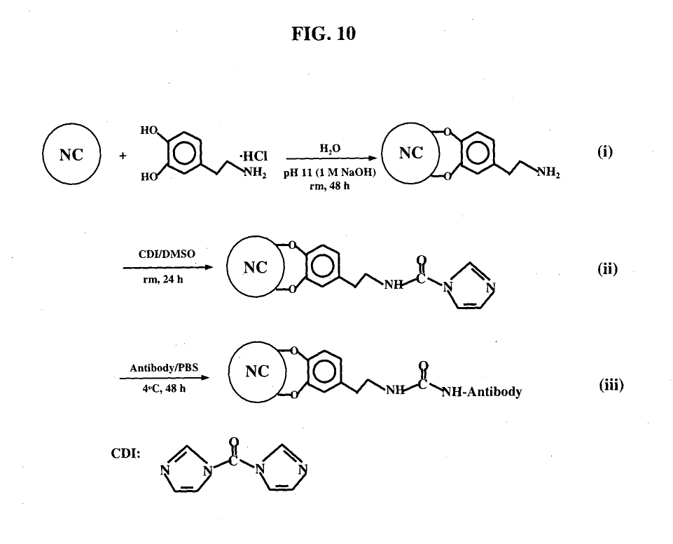

FIG. 10 shows formation of a nanoconjugate according to various embodiments of the present invention. The conjugation reaction involves three steps, as shown in FIG. 10: i) catecholic L-3,4 dihydroxy-phenylalanine (dopamine), a major component of natural glue proteins secreted by mussels, was firstly anchored onto the nanocomposites (NCs) surfaces to provide active amino groups. DOPA has previously been shown to be able to anchor functional biomolecules and polymers onto a large variety of surfaces, including metals, metal oxides and glasses; ii) a bifunctional cross-linking agent, 1,1′-carbonyldiimidazole (CDI), was coupled to the NCs to allow further active sites for anchoring of antibody. The CDI was used instead of using due to its good biocompatibility. CDI is often used for the coupling of amino acids for peptide synthesis and as a reagent in organic synthesis; iii) the antibody was conjugated onto the NCs surface, resulting in antibody-conjugated NCs.

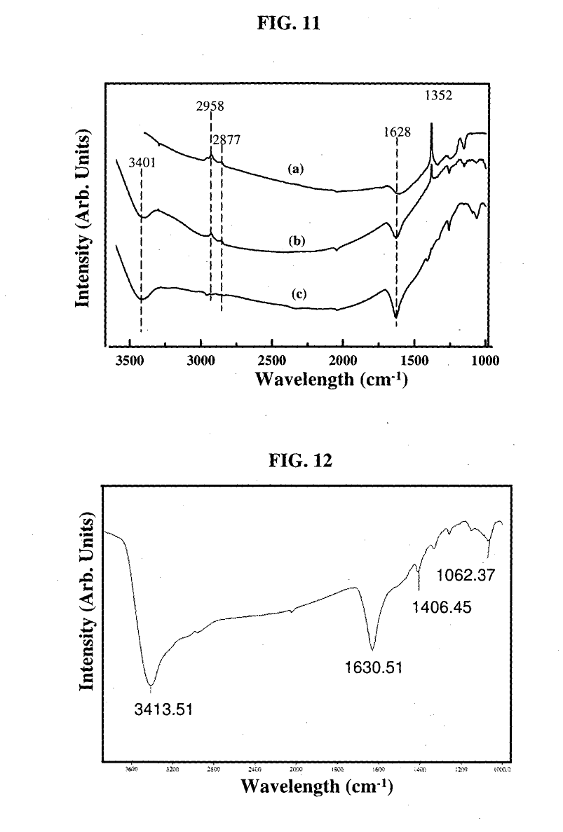

FIG. 11 shows the fourier transform infrared (FTIR) spectra of (a) Dopamine-immobilized nanocomposites from end of step (i) in FIG. 10 (b) CDI-immobilized nanocomposites from end of step (ii) in FIG. 10 and (c) antibody-conjugated nanocomposites (nanoconjugates) according to various embodiments of the present invention from end of step (iii) in FIG. 10. The additional peak at 3401 cm⁻¹, attributed to the O—H and N—H stretching vibrations, can be observed on the CDI-immobilized and antibody-conjugated NCs. The characteristic peaks of the C—H asymmetric and symmetric stretching vibration, at 2958 and 2877 cm⁻¹ respectively, is invisible on the antibody-conjugated NCs. The peak at 1628 cm⁻¹ is associated with the amide C═O stretching vibration (amide I) band.

FIG. 12 shows the fourier transform infrared (FTIR) spectra of antibody-conjugated nanocomposites (nanoconjugates) according to various embodiments of the present invention. The peaks at 3413 cm⁻¹ (attributed to the O—H and N—H stretching vibration) and 1630 cm⁻¹ (attributable to the amide I stretching vibration) are the characteristics of the antibody. Furthermore, the increase in the relative intensity of these peaks indicates the increase of the amount of conjugated antibody on the nanocomposites' surface. Therefore, FIG. 12 shows that the antibody has been successfully conjugated onto nanocomposites using the three-step covalent reaction processes in FIG. 10.

In a third aspect, the present invention relates to a method for killing cells including contacting said cells with the composite material or the conjugate, and irradiating the cells and the composite material or the conjugate with NIR radiation. The cells may be cancer cells.

In a fourth aspect, the present invention relates to a method for treating cancer in a subject, including delivering the composite material or conjugate material to said subject and irradiating the subject or part of the subject with NIR radiation.

In a fifth aspect, the present invention relates to a use of the composite or conjugate for the killing of cells.

In a sixth aspect, the present invention relates to a use of the composite or conjugate for the treatment of cancer in a subject.

In various embodiments, the reactive species generated by the composite material upon irradiation can help to kill cells such as cancer cells. In other words, the method of killing cells may include irradiating the cells and the composite material or the conjugate with NIR radiation such that the reactive species are generated by the composite material or the conjugate to kill the cells. The use of the composite or conjugate for killing cells may include generating reactive species by the composite or conjugate upon NIR irradiation to kill the cells. The use of the composite or conjugate for the treatment of cancer in a subject may include irradiating the subject or part of the subject with NIR irradiation such that the composite or conjugate generates reactive species to kill cancer cells. The reactive species may be generated by the composite material from water or other suitable molecules using the composite material as a catalyst.

In various embodiments, the reactive species are reactive oxygen species (ROS) such as radicals including O. and OH. as well as superoxide anions (O₂.), hydrogen peroxide and singlets oxygen (¹O₂). The reactive oxygen species (ROS) may be formed from water using the composite material as a catalyst under irradiation.

It may be required to control the frequency and dosage delivered to the human or animal body so as to reduce damage to healthy cells near the cancer cells. The physiologically acceptable composite material may be delivered to the human or animal body through ingestion, injection or other means in a relatively safe manner. NIR can penetrate into tissues to activate the composite material to generate reactive species. As such, by controlling the amount and frequency of NIR irradiation, the release of reactive species to cancer cells in the human or animal body can be controlled.

In various embodiments, an antibody, drug or therapeutic substance may be attached to the composite material to form a conjugate. The antibody, drug or therapeutic substance may be attached to the composite material through one or more coupling agents. When the conjugate is irradiated by NIR, the reactive species generated cleave a covalent link between the composite and the antibody/drug/therapeutic substance or between the coupling agents and the antibody/drug/therapeutic substance or a link within the coupling agents. The antibody/drug/therapeuctic substance is thus released to target the cells. The antibody may be a cancer targeting antibody such as anti-cAngpt14 to target cancer cells. In other words, the method of killing cells may include irradiating the cells and the composite material or the conjugate with NIR radiation such that reactive species are generated by the composite material or the conjugate to cleave a covalent link between the composite and the antibody or between the coupling agents and the antibody or a link within the coupling agents. The use of the composite or conjugate for killing cells may include generating reactive species by the composite or conjugate upon NIR irradiation to release antibodies to kill the cells. The use of the composite or conjugate for the treatment of cancer in a subject may include irradiating the subject or part of the subject with NIR irradiation such that the composite or conjugate generates reactive species to release anti-cancer drugs or antibodies or therapeutic substance to kill cancer cells.

By using a conjugate comprising an antibody, drug or therapeutic substance attached to the composite material, a more controlled release of the antibody drug or therapeutic substance using the photocatalytic property of the semiconductor particulate material may be achieved. The conjugate may be delivered to the human or animal body through ingestion, injection or other means. NIR can penetrate into tissues to activate the conjugate to release the antibody, drug or therapeutic substance. As such, by controlling the amount and frequency of NIR irradiation, the release of antibodies, drugs or therapeutic substances in the human or animal body can be controlled.

Advantageously, a method for killing cells including contacting said cells with the composite material or the conjugate, and irradiating the cells with NIR radiation or a use of the composite or conjugate for the killing of cells or a use of the composite or conjugate for the treatment of cancer in a subject, wherein the composite or conjugate is physiologically acceptable and wherein NIR radiation is upconverted to visible light helps to reduce the damage caused to nearby healthy cells.

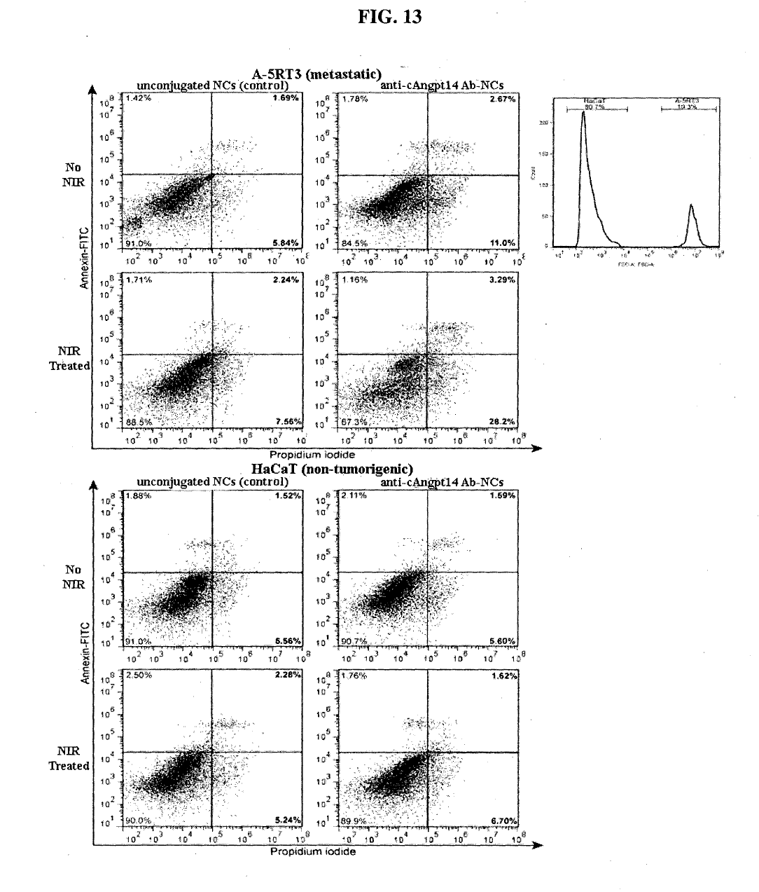

FIG. 13 show florescence activated cell sorting (FACS) analysis when cells are treated with anti-cAngpt14 conjugated with nanocomposites to form nanoconjugates according to various embodiments of the present invention. The analysis show an increase in apoptotic A-5RT3 cells (Abbexib V⁺/PI⁺ and Annexin V⁺/PI⁻) when treated with anti-cAngpt14 antibody (Ab)-nanoconjugates when compared with nanocomposites without anti-cAngpt14 antibodies, even in the absence of NIR exposure (unconjugated vs anti-cAngpt14-conjugated: 7.53% vs 13.67%). Upon NIR exposure, anti-cAngpt14 Ab-nanoconjugates treated A-5RT3 showed a further ˜2.5-fold increase (31.49%) in apoptotic cells. Although there was a slight increase in the percentage of apoptotic cells in NIR exposed unconjugated nanocomposites treated A-5RT3 (9.8%), this difference was not statistically significant. FACS analysis also showed no significant difference in the percentage of apoptotic HaCaT cells treated with unconjugated nanocomposites regardless of NIR exposure, suggesting that the nanocomposites exerted their cytotoxic effects only in close proximity to the cells. The functionalization of the N nanocomposites Cs with anti-cAngpt14 antibody conferred selective anti-tumor property. The experiments were repeated except that HaCaT were prelabelled and similar results were obtained.

Other embodiments are within the following claims and non- limiting examples.

### EXAMPLES

Materials: Ethylene glycol (EG, 99%), NaCl (99%), YCl₃.6H₂O (99.9%), YbCl₃.6H₂O, TmCl₃.6H₂O, Branched polyethyleminine (PEI, 25 KDa), thioglycolic acid, titanium n-butoxide, HNO₃ solution (69%), acetyl acetone, NH₄F (99%), terephthalic acid (99%), NaOH (99%), 7-methoxycournarin-3-carboxylic acid, isopropanol, toluene, triethylamine, dimethyl sulfoxide, 3-Aminopropyltriethoxysilane.

### Example 1

### Preparation of Yb, Tm-doped NaYF₄ Nanoparticles (NaYF₄:Yb,Tm)

1.2 mmol of NaCl, 0.48 mmol of YCl₃.6H₂O, 0.108 mmol of YbCl₃.6H₂O 1 μmol of TmCl₃.6H₂O and 0.15 g of PEI were dissolved in 9 mL EG solvent. The mixture solution was dropped into a stoichiometric amount of NH₄F in 6 mL of EG. The resulting mixture was agitated for another 10 min, then transferred to a 20 mL Teflon-lined autoclave, and subsequently heated at 200° C. for 2 h. Acetone was added into the obtained solution and the NaYF₄:Yb,Tm nanoparticles were collected by centrifugation. Finally, the NaYF₄:Yb,Tm was washed with ethanol and DI water for several times, and dispersed in DI water at concentration of 1.0 wt. %.

### Example 2

### Preparation of N-Doped TiO₂ Nanoparticles (N—TiO₂) and Thioglycolic Acid Functionalized N—TiO₂ Nanoparticles (TGA-N—TiO₂)

Pure N—TiO₂ nanoparticles were prepared by a hydrothermal reaction. Typically, a mixture of 5.0 mL of titanium n-butoxide and 5.0 mL of isopropyl alcohol was added dropwise into 30 mL HNO₃ solution (0.2 M) containing 1.0 mL of acetyl acetone, and kept continuous stirring for 12 h. After that, 5.0 mL of triethylamine was added into the mixture solution and kept continuous stirring for another 12 h. Then, the mixture was put into a Teflon-lined stainless autoclave and hydrothermally treated at 160° C. for 12 h. The powder was filtered, washed with DI water five times. The obtained N—TiO₂ nanoparticles were treated with thioglycolic acid (TGA) at room temperature and kept stirring continuous for 3 h. After that the TGA-N—TiO₂ was washed with DI water for several times and dispersed on DI water at concentration of 1.0 wt. %.

### Example 3

### Preparation of N—TiO₂/NaYF₄:Yb,Tm Nanocomposites

The NaYF₄:Yb,Tm and TGA-N—TiO₂ (w/w=2/1) were mixed in 50 mL DI water and heated at 160° C. for 3 h. Then the N—TiO₂/NaYF₄:Yb,Tm was collected by centrifugation (10000 rpm, 5 min) and washed with DI water for several times. Finally, the obtained yellow powder was dried in an oven at 70° C. for 12 h.

### Example 4

### Detection of Photogenerated OH Radicals

Typically, 10 mg of N—TiO₂/NaYF₄:Yb,Tm was added in 5 mL mixture solution of terephthalic acid (8×10⁻⁴ M) and NaOH (4×10⁻⁴ M). The mixture was put in an ultrasonic for 30 min to disperse the N—TiO₂/NaYF₄:Yb,Tm uniformly in the solution. Then the mixture was irradiated with a NIR laser (power=3 W and λ=980 nm). At every 30 min, 0.5 mL of the suspension was collected and centrifuged (10000 rpm, 5 min). Then 0.3 mL of the transparent solution was diluted 20 times for the PL measurement. The concentration of hydroxyterephthalate anion was measured by fluorescence analysis (Fluoromax-4 Spectrophotometer, Horiba Jobin Yvon) with an excitation wavelength of 320 nm. Pure N—TiO₂, NaYF₄:Yb,Tm and blank were also analyzed under the same conditions for comparison.

### Example 5

### Photocatalytic Activities Measurement

The photocatalytic activities of the N—TiO₂/NaYF4:Yb,Tm, N—TiO₂ and NaYF₄:Yb,Tm were measured by the degradation of methylene blue (MB) in an aqueous solution. 10 mg of sample was suspended in a 5 mL aqueous solution of MB (10 ppm). Prior to irradiation, the suspension was stirred in the dark for 24 h to establish an adsorption/desorption equilibrium between the photocatalyst and MB. Then the mixture was irradiated with a NIR laser (BWOF-2, B&W TEK Inc., power=2 W and λ=980 nm) and the concentration of MB was determined (according to the concentration-absorbance at λ=664 nm relationship) at 6 hours time interval up to 30 hrs.

### Example 6

### Attachment of Fluorescent Dye on N—TiO₂/NaYF₄:Yb,Tm

A modified method was used to attach the fluorescent dye on the N—TiO₂/NaYF₄:Yb,Tm. Firstly, the N—TiO₂/NaYF₄:Yb,Tm was refluxed in 10 mM 3-Aminopropyltriethoxysilane (APTES)-toluene solution for 24 h at 70° C., which led to a saturated APTES monolayer on the surface of the N—TiO₂/NaYF₄:Yb,Tm. Then the APTES-N—TiO₂/NaYF₄:Yb,Tm was collected by centrifugation (10000 rpm, 5 min) and washed with dimethyl sulfoxide (DMSO) for several times. After that the APTES-N—TiO₂/NaYF₄:Yb,Tm was refluxed in fluorescent dye (7-methoxycoumarin-3-carboxylic acid)-DMSO solution for 2 h at 70° C. Finally, yellow precipitate was collected by centrifugation (10000 rpm, 5 min), cleaned by immersing in DMSO for 30 min and dried at 70° C. for 24 h.

### Example 7

### NIR-Induced Release of Dye

14.0 mg of fluorescent dye-modified N—TiO₂/NaYF₄:Yb,Tm was suspended in a 3.5 mL DI water in a quartz cuvette. The mixture was put in an ultrasonic for 30 min to disperse the powder uniformly in the solution. Then the mixture was irradiated with a NIR laser (BWOF-2, B&W TEK Inc. power=2 W and λ=980 nm). After 10 min, 3.0 mL of the suspension was collected and centrifuged (10000 rpm, 5 min). The transparent solution was diluted 20 times for the PL measurement with an excitation wavelength of 320 nm. In order to confirm that no dye was released without NIR irradiation, 7.0 mg of fluorescence dye-modified N—TiO₂/NaYF₄:Yb,Tm was immersed in a 3.5 mL DI water for 120 mM. After removing the powder, the water was irradiated with UV laser, but no fluorescence was detected.

### Example 8

### Characterization

X-ray diffraction analysis (XRD) was carried out using a Philips PW1010 X-ray diffractometer with Cu K_{α} radiation. XRD pattern was recorded with a scan step of 1° min⁻¹ (2θ) in the range from 20° to 70°. Surface species of these samples were analyzed by Fourier Transform Infrared (FTIR) Spectroscopy (Digilab FTS 3100). X-ray photoelectron spectroscopy (XPS) analysis was carried out with a PHI Quantum 2000 Scanning ESCA Micro-probe equipment (Physical Electronics, MN, USA) using monochromatic Al-Kα radiation. The X-ray beam diameter was 100 μm, and the pass energy was 29.35 eV for the sample. The binding energy was calibrated with respect to C (1s) at 284.6 eV. The high resolution transmission electron microscopy (HRTEM), transmission electron microscopy (TEM) was acquired using a JEOL JEM-2100F microscope operating at 200 kV. UV-Vis-NIR diffuse reflectance spectra (DRS) were obtained using a CARY 5000 UV-Vis-NIR spectrophotometer (VARIAN).

### Example 9

### Surface Conjugation Reaction

In step 1, the nanocomposites (NCs) were immersed in a 2 mg/ml aqueous solution of dopamine for 48 h in the dark using aluminum foil. To prevent the protonation of the amine groups, the reaction mixture was adjusted to pH 11 using 1 M NaOH. At the end of the reaction, the NCs were harvested by centrifugation at 8000 rpm for 45 minutes, and followed by re-suspension in copious amount of deionized water for twice to remove the unattached dopamine, and finally were dried under vacuum conditions overnights.

In step 2, the dopamine-immobilized NCs were immersed in a 10 mg/mL DMSO solution of CDI at room temperature for 24 h in the dark using aluminum foil. At the predetermined reaction time, the resulting NCs were harvested by centrifugation at 8000 rpm for 45 minutes, followed by re-suspension in copious amounts of tetrahydrofuran (THF) and deionized water, respectively, and finally dried under vacuum conditions overnights.

In step 3, the CDI-immobilized NCs were immersed in a phosphate buffered saline (PBS) (pH=7.4) solution of antibody for 48 h at 4° C. in the dark using aluminum foil. After the reaction, the NCs were harvested by centrifugation at 3000 rpm for 60 minutes, followed by re-suspension in phosphate buffered saline (PBS) solution twice to remove the physically-absorbed antibody, and finally dried in freeze-drier for 24 h. Three batches of antibody-conjugated NCs were completed using the conjugation reaction conditions shown in Table 1.

TABLE 1

| | | | Reaction |
|---|---|---|---|
| Antibody conc | Volume | Temperature | time |
| (μg/mL) | (mL) | (° C.) | (h) |
| 10 | 1.5 | 4 | 48 |
| 50 | 3 | 4 | 72 |
| 100 | 3 | 4 | 72 |

### Example 10

### Cell Culture

Metastatic human squamous cell carcinoma A-5RT3 and non-tumorigenic human keratinocyte cell line HaCaT are routinely cultured as a monolayer in DMEM (Dulbecco's modified Eagle's medium) supplemented with 10% fetal bovine serum. The culture is kept at a 5% CO₂ humidified incubator at 37° C. Standard trypsinization procedure is used for subculturing.

### Example 11

### Apoptosis Assay

HaCaT cells were cocultured with A-5RT3 cells prelabeled with CellTracker Blue CMAC (7-amino-4-chloromethylcoumarin; Life Technologies) at 4:1 ratio in each well of 24-well petri dish. The cells (2×10⁵) were incubated overnight to allow attachment to the plate. Following day, the medium was replaced with serum-free phenol-red DMEM. Triplicate samples of cells were allowed to react with either unconjugated or anti-cAngpt14 Ab-conjugated nanoparticles at a concentration ˜250 ng/ml for 1 hour before exposure to far-IF source at 2 Amp for 120 sec. The cells in each well were trypsinized and apoptotic cells detected by Annexin V/PI staining followed FACS analysis as described by manufacturer (BioLegends). The two cell types were distinguished based on CellTracker dye.

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

By “about” in relation to a given numerical value, such as for temperature and period of time, it is meant to include numerical values within 10% of the specified value.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non- limiting examples. In addition, where features or aspects of the invention are described in terms of Markush groups, those skilled in the art will recognize that the invention is also thereby described in terms of any individual member or subgroup of members of the Markush group.

1. A method for killing cancer cells in a subject, the method comprising contacting said cancer cells with a composite material or a conjugate, the conjugate comprising the composite material and at least one compound covalently linked to the composite material; and
irradiating the composite material or the conjugate with near infrared radiation (NIR) radiation to release reactive species to kill the cancer cells in the subject;
wherein the composite material comprises:
at least one upconversion particulate material that under near infrared radiation emits visible light of a wavelength between 380 and 740 nm; and
at least one semiconductor particulate material that can absorb the visible light emitted by the at least one upconversion particulate material and upon absorbance generates the reactive species; wherein the at least one upconversion particulate material and the at least one semiconductor particulate material are physiologically acceptable;
wherein the at least one upconversion particulate material comprises or consists of NaYF₄ doped with at least one rare earth metal; and
wherein the at least one semiconductor particulate material comprises or consists of TiO₂ doped with another element.

2. The method according to claim 1,
wherein the at least one upconversion particulate material and the at least one semiconductor particulate materials are bonded to each other.

3. The method according to claim 2,
wherein the at least one upconversion material particulate material is bonded to the at least one semiconductor particulate material via at least one linker molecule.

4. The method according to claim 1,
wherein the at least one rare earth metal is selected from the group consisting of Sc, Y, La, Ce, Pr, Nd, Pm, Sm, Eu, Gd, Tb, Dy, Ho, Er, Lu, Yb, Tm and combinations thereof.

5. The method according to claim 4, wherein the upconversion particulate material is NaYF₄:Yb; NaYF₄:Tm or NaYF₄:Yb, Tm.

6. The method according to claim 1,
wherein the dopant is selected from the group consisting of N, P, C, B, S, Fe, Ag, Au, Ce, Er, Eu or any other suitable elements and combinations thereof.

7. The method according to claim 1,
wherein the reactive species generated by the at least one semiconductor particulate material is a reactive oxygen species (ROS).

8. The method according to claim 1
wherein the composite material is a nanocomposite material.

9. The method according to claim 1,
wherein the composite material and at least one compound covalently linked to the composite material via a covalent link; and
wherein the reactive species generated cleave the covalent link to release the compound to kill the cancer cells.

10. The method according to claim 1
wherein the at least one compound is selected from the group comprising dyes, proteins, peptides and drugs.

11. The method according to claim 10, wherein the drug is a small molecule drug or an antibody.

12. The method according to claim 10, wherein the drug is an anti-cancer drug.

13. The method according to claim 1,
wherein the conjugate is a nanoconjugate.

14. The method according to claim 1,
wherein amount and frequency of near infrared irradiation is controlled to kill the cancer cells.

15. A method for treating cancer in a subject, comprising
delivering a composite material or a conjugate, the conjugate comprising the composite material and at least one compound covalently linked to the composite material to said subject; and
irradiating the subject or part of the subject with near infrared (NIR) radiation to release reactive species to kill cancer cells in the subject;
wherein the composite material comprises:
at least one upconversion particulate material that under near infrared radiation emits visible light of a wavelength between 380 and 740 nm; and
at least one semiconductor particulate material that can absorb the visible light emitted by the at least one upconversion particulate material and upon absorbance generates the reactive species; wherein the at least one upconversion particulate material and the at least one semiconductor particulate material are physiologically acceptable;
wherein the at least one upconversion particulate material comprises or
consists of NaYF₄ doped with at least one rare earth metal; and
wherein the at least one semiconductor particulate material comprises or consists of TiO₂ doped with another element.

16-17. (canceled)

18. Composite material for killing cancer cells, the composite material comprising:
at least one upconversion particulate material that under near infrared (NIR) irradiation emits visible light of a wavelength between 380 and 740 nm; and
at least one semiconductor particulate material that can absorb the visible light emitted by the at least one upconversion particulate material and upon absorbance generates reactive species to kill the cancer cells; wherein the at least one upconversion particulate material and the at least one semiconductor particulate material are physiologically acceptable;
wherein the at least one upconversion particulate material comprises or consists of NaYF₄ doped with at least one rare earth metal; and
wherein the at least one semiconductor particulate material comprises or consists of TiO₂ doped with another element.

19. The composite material according to claim 18, wherein the at least one upconversion particulate material and the at least one semiconductor particulate materials are bonded to each other.

20. The composite material of claim 19, wherein the at least one upconversion material particulate material is bonded to the at least one semiconductor particulate material via at least one linker molecule.

21. A conjugate comprising
the composite material according to claim 18; and
at least one compound covalently linked to the composite material.

22. The conjugate of claim 21 wherein the at least one compound is selected from the group comprising dyes, proteins, peptides and drugs.
